(19) United States
(12) Patent Application Publication
(10) Pub. No.: US 2004/0163643 A1
(43) Pub. Date: Aug. 26, 2004

(54) Pocket vaporizer

(76) Inventor: Ben Carroll Wesner, Ukiah, CA (US)

Correspondence Address: Ben Wesner, Suite A, 1111 North Palm Canyon Drive, Palm Springs, CA, 92262, US

(21) Appl. No.: 10/371,580

(22) Filed: Feb. 24, 2003

(51) Int. Cl. A61M 15/06

(52) U.S. Cl. 128/202.21

(57) ABSTRACT

A vaporizer apparatus and method of extracting active ingredients of a specimen of crude natural product, without pyrolysis, by applying heat to the exterior of the apparatus directly. The chamber of the apparatus is held above a low temperature heat source, usually an open flame. The apparatus is manually turned to distribute the heat over the chamber. Through radiation, the essential oils and medicines contained in the natural herb are subsequently volatized and collected within the chamber and are inhaled through the mouth of the apparatus.

### CROSS-REFERENCE TO RELATED APPLICATIONS

### Not Applicable

### FEDERALLY SPONSORED RESEARCH

### Not Applicable

### SEQUENCE LISTING OR PROGRAM

Not Applicacble

### BACKGROUND OF THE INVENTION

### Field of Invention

The present invention relates to a vaporizer for inhalation and a method for extraction of active ingredients from a crude natural product, by means of heating the apparatus to the temperature appropriate to volatilize the molecules.

### BACKGROUND OF THE INVENTION

The use of plants as medicinal agents has a long and successful history. The majority of medicines used today are either derived directly from plants or synthesized as variations on natural molecules. However, modem medical practice has, for the most part, abandoned the use of whole plant products because of objections concerning variability in concentration of active ingredients, and unpredictable rates of active drug release from orally ingested crude drugs. Even teas made from medicinal plants cannot fully overcome the latter objection, as the dose provided depends on compound solubility and the conditions of extraction. In addition, accurate self-titration of this dose cannot be expected to overcome either objection, considering the excessive time-lag between oral ingestion and the onset of action.

The best resolution of these problems is through pulmonary ingestion of vaporaized compounds, if they are sufficiently volatile. This provides a more immediate means of relief and a more accurate method for dose self-titration, as well as alllowing a means for applying compounds to the pulmonary tract itself, as is necessary with diseases such as bronchial asthma, etc;. Pulmonary ingestion of drugs also circumvents the “first-pass effect” by which oral drugs are transferred from the intestines and then partially or entirely metabolized by the liver, before entering the blood stream.

Unfortunately, the only technique available to accomplish pulmonary application of crude natural drugs has been via the method of smoking. This is objectionable from the medical perspective because pyrolysis products are irritating and long-term ingestion of smoke has been implicated in the etiology of various pulmonary disease states (e.g., emphysema, cancer, etc;).

Other objections to inhalers such as U.S. Pat. No. 87,603 (Tichenot) which continuously heats or pyrolizes a substance on a grating, and U.S. Pat. No. 1,858,580 (Collins) which steam heats a carrying agent permeated with a medicated substance, involve the lack of ability to either maximize the extraction exposure of the specimen particles, or to promote a uniform extraction of the aggregate charge through its periodic mixing. In addition, inhalers such as U.S. Pat. No. 6,250,301, (Pate) require a heated airstream which increases the cost of the inhaler, limits its portability, and is not necessary when using radiant heat to achieve volatilization of the substance. A uniform extraction of the aggregate charge is achieved through the manual rotation of the apparatus.

### SUMMARY OF THE INVENTION

An object of the present invention is to provide a vaporizer using low temperature radiant heat and manual rotation of the apparatus to vaporize and uniformly extract the active ingredients of a crude natural product or products without pyrolysis, thereby avoiding the potentially detrimental effects of smoking.

A further object of the present invention is to spare the wasteful loss of active ingredients extracted by the vaporizer due to a continuous gas flow or continuous radiant heating instead of a demand-only radiant heating of the apparatus.

The present invention extracts volatile active ingredients from a crude natural product, by means of radiant heat at the appropriate temperature to volatilize the molecules of the product or substance. Transfer of these agents or ingredients as a water-free aerosol is accomplished by inhaling through the mouthpiece. In addition, manually rotating the apparatus provides for a uniform heat distribution causing a mixing of the aggregarte charge upon each inhalation.

A heat source is applied to the exterior of the apparatus, usually a butane lighter or candle. As the temperature of the flame-resistant glass chamber increases, radiant heat transfer causes the specimen particles to be suspended within the confines of the isolation chamber.

By removing the external heat source, the temperature of the glass chamber decreases, preventing wasteful loss of active ingredients. The concavity of the apparatus at the chamber opening further prevents pyrolysis and the loss of ingredients.

### BRIEF DESCRIPTION OF THE DRAWINGS

Other features and advantages of the invention will be apparent from the following description taken in connection with the accompanying drawings, wherein:

FIG. 1 shows the completed assembly of the vaporizer apparatus according to a first embodiment of the invention.

FIG. 2 shows the isolation chamber of the vaporizer apparatus.

FIG. 3 shows the hollow cylindrical mouthpiece of the vaporizer apparatus.

FIG. 4 shows the restriction in the inner wall of the vaporizer apparatus which separates the isolation chamber from the mouthpiece.

FIG. 5 shows the chamber opening of the vaporizer apparatus where product is inserted.

FIG. 6 shows the mouthpiece opening of the vaporizer apparatus where transfer of the ingredients occurs through inhalation.

FIG. 7 shows the concavity of the chamber opening of the vaporizer apparatus to further prevent pyrolysis and loss of ingredient.

FIG. 8 shows the feet of the vaporizer apparatus which stabilize the invention when resting on a flat surface and creates a heat barrier between the isolation chamber and the mouthpiece.

### DETAILED DESCRIPTION OF THE DRAWINGS

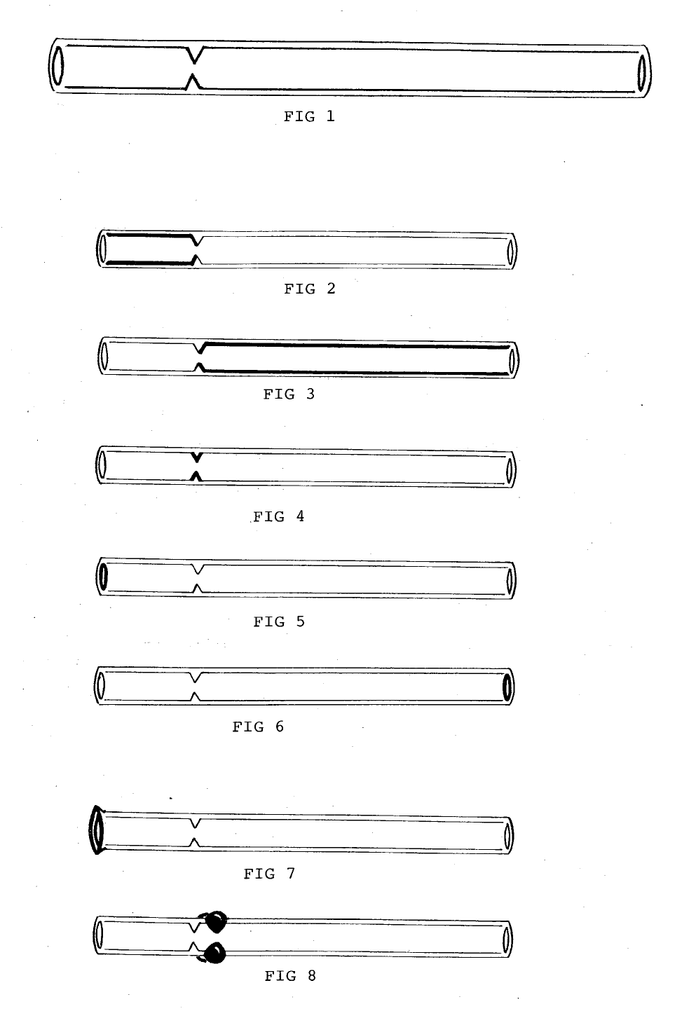

A completed assembly of the first embodiment of the vaporizer apparatus of the present invention is shown in FIG. 1. The entire vaporizer apparatus is made out of boro-silicate glass. The overall length is roughly from 2 inches to 12 inches. The exterior diameter is roughly from 1/4 inch to 2 inches. The interior diameter is roughly from 1/16 inch to 1 inch. The mouthpiece of the vaporizer apparatus, FIG. 3 (roughly 1/2 inch to 11 inches in length), is held by the user between two fingers. The specimen particles are inserted into the isolation chamber, FIG. 2 (roughly 1/4 inch to 3 inches in length) through the chamber opening, FIG. 5 (roughly 1/8 inch to 1 inch in diameter). The isolation chamber, FIG. 2, is held directly over an open flame, provided by a portable heat source, such as a butane lighter or candle.

Once the isolation chamber, FIG. 2, is heated to the proper temperature, the volatile components of the specimen becomes vaporized, through radiant heat transfer. This vapor is drawn into the pulmonary tract, by inhaling through the mouthpiece opening, FIG. 6 (roughly 1/16 inch in diameter).

The restriction in the inner wall of the vaporizer apparatus, FIG. 4 (creates an opening roughly from 1/16 inch to 1/4 inch in diameter), prevents large particles of the substance from entering into the mouthpiece. FIG. 7 shows the concavity of the chamber opening of the vaporizer apparatus to further prevent pyrolysis and loss of ingredients. FIG. 8 shows the feet of the vaporizer apparatus which act to stabilize the invention when resting on a flat surface and, more importantly, act as a heat barrier between the isolation chamber and the mouthpiece to prevent the mouthpiece from becoming too hot to hold and manipulate manually.

It is contemplated that numerous modifications may be made to the appartus and procedure of the invention without departing from the spirit and scope of the invention as described in the drawings and these descriptions.

What is claimed is:

1. A hand held vaporizer apparatus, comprising:
a) a flame-resistant isolation chamber for the containment of a crude natural substance;
b) a mouthpiece for holding the vaporizer between the fingers and for inhaling the vapors after heating the isolation chamber to the appropriate temperature.
